(19) United States
(12) Patent Application Publication
(10) Pub. No.: US 2015/0342494 A1
(43) Pub. Date: Dec. 3, 2015

(54) SPORTS DEVICE AND SYSTEM

(71) Applicant: SENSILK INC., San Francisco, CA (US)

(72) Inventors: Iou-Ming Lou, Taipei (TW); Takuang Yang, Palo Alto, CA (US); Mary Bernadette Vincent, Newark, CA (US)

(73) Assignee: SENSILK INC., San Francisco, CA (US)

(21) Appl. No.: 14/303,604

(22) Filed: Jun. 13, 2014

Related U.S. Application Data
(60) Provisional application No. 61/918,675, filed on Dec. 20, 2013.

(51) Int. Cl. A61B 5/0408; A61B 5/00

(52) U.S. Cl. CPC A61B 5/04085; A61B 5/6804; A61B 5/0022; A61B 5/746; A61B 2562/0209

(57) ABSTRACT

A device is designed for collecting heartbeat information. Heart Beat Variation (HRV) is calculated based on the collected heartbeat information. The device has textile sensors so that a user wears a clothing with such sensors to collect the heartbeat information.

### FIELD OF THE INVENTION

The present invention relates to sports device and system and more particularly relates to sports device and system that provide information to users.

### BACKGROUND

Along with electronic technology development, more and more electronic devices of various applications are invented for enhancing human life quality. In addition, there are more and more flexible protocols for electronic devices to communicate to each other.

Still, there are many possibilities to figure out even more new technologies to satisfy unlimited human needs for new tools to make life better.

### DESCRIPTION OF DRAWINGS

FIG. 1 illustrates an embodiment of the present invention;

FIG. 2 illustrates an exemplary ECG diagram;

FIG. 3 illustrates HRV;

FIG. 4 illustrates several embodiments of the present invention;

FIG. 5A, FIG. 5B. FIG. 5C and FIG. 5D illustrate bra device examples;

FIG. 6A and FIG. 6B illustrate bra device examples;

FIG. 7A, FIG. 7B and FIG. 7C illustrate bra device examples;

FIG. 8 illustrates an embodiment of textile sensors;

FIG. 9 illustrates exemplary arrangement of sensor elements;

FIG. 10 illustrates an embodiment of the present invention; and

FIG. 11 illustrates a method according to an embodiment.

### DETAILED DESCRIPTION

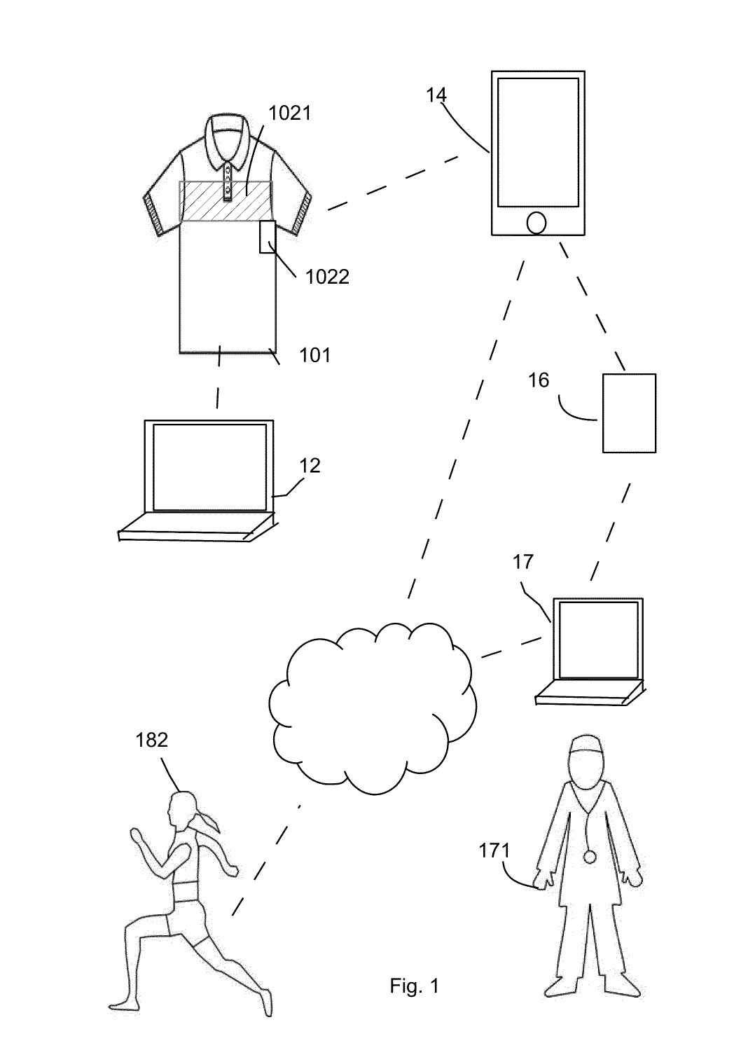

FIG. 1 illustrates a first embodiment according to the present invention. A shirt 101 is attached with a textile sensor 1021. The textile sensor 1021 is coupled to a signal processor 1022. The textile sensor 1021 contains conductive fiber woven interleaved within insulation material fiber. The conductive fiber has a plurality of contact parts capable of contacting chest of a user when the user wears the shirt. The contact parts may sense physiological signals spreading on the surface of the chest of the user, and convert the physiological signals into minor electronic signals to be collected, processed, and analyzed. This is similar to ECG (Electrocardiography) detection seen in hospitals and the difference is that the electrodes used in hospitals are now made of fiber and woven into clothing.

Such minor electronic signals are transmitted to the signal processor 1022. The signal processor 1022 amplifies, analyzes, filters, compresses, stores and/or transmits collected signals for further analysis and use in various applications. The signal processor 1022 may have an analog to digital converter (not shown) to turn collected analog signals into digital signals. When the analog signals are converted into digital signals, there are various ways to process the signals. For example, ECG waves may be constructed, and/or Fourier Transformation may be applied to analyze their spectrum characteristics. Raw waveforms may be stored, or only certain characteristics are captured while other data are skipped for saving power. Further processing possibilities are discussed below.

The signal processor 1022 may also have a wireless and/or a wire transmission interface for connecting to other devices. For example, the signal processor 1022 may be disposed together with a Bluetooth transmitter, a Blue tooth receiver, a Bluetooth transceiver, an NFC(Near Field Communication) transmitter, an NFC receiver, an NFC transceiver, a Wi-Fi transceiver, and/or other communication components following standard protocols like ANT or other proprietary protocols.

Via such transmission interface, physiological signals collected by the textile sensor 1021 may be transmitted to a mobile phone 14, a computer 12, a television (not shown), a tablet pad (not shown), a wireless storage (not shown), another wearable electronic device (e.g., smart watch, smart bracelet, smart glasses, smart wristband, smart jewelry, or smart apparel . . . etc.) and/or other devices. When a mobile phone 14 is used, due to current powerful computing capability, raw data may be further analyzed, integrated with other information, like where the user is located, to determine current scenario, schedule information recorded on the mobile phone 14 to determine what event is handling by the user, e.g. in a gym, and/or other useful information. In addition, useful information may be feedback instantly to the user via visual information like graphs, light color, etc., audio information, vibration, and any combination thereof. Alternatively, the data may be collected for a period of time and then uploaded to the mobile phone 14 for batch processing.

In addition to synchronously transmitting collected signals, a storage, e.g. a memory module or a detachable memory card, may be used for storing data temporarily before collected signals are transmitted to other devices. Besides, the signal processor and/or other related component may be designed as a detachable or removable component so that users may move the signal processor to another shirt or use another signal processor to put on the old shirt for meeting different needs.

A cradle or other storage device may also be designed for users to conveniently store these components like signal processor and/or the textile sensor when they are removed from the shirt.

As shown in FIG. 1, collected physiological information by the textile sensor 1021, under different designs, may be transmitted at the same time to multiple devices, like the mobile phone 14 and the computer 12. Generally, the computer 12 may have more powerful capability, and complex analysis and processing may be handled.

In another aspect, there are various ways for the signal processor 1022 co-working with the mobile phone 14 or other external devices. For example, the signal processor 1022 may be designed to have multiple modes. When the signal processor 1022 is matched and connected to the mobile phone 14, the signal processor 1022 may send raw data directly to the mobile phone 14 for further processing to save power consumed by the signal processor 1022. In other words, certain processing originally handled by the signal processor 1022 is now handled by the mobile phone 14. When the signal processor 1022 is not matched and connected to an external device like the mobile phone 14, the signal processor 1022 may enter into other modes. For example, the signal processor 1022 may shut down transmission handling to save power and focus on storing data for later being uploaded to another external device.

When the signal processor 1022 is not connected to an external device, the signal processor 1022 may be designed to store only the most-updated information. Alternatively, the signal processor 1022 may be designed to use more sophisticated rules to make the most of its limited storage capacity. For example, the signal processor 1022 may selectively store only important information and ignore other information instead of always storing the most-updated information while excluding the oldest information. For example, the signal processor 1022 may identify certain predetermined patterns, and use such patterns for determining how to store corresponding information. For example, some heart disease patterns or common patterns related to the user or related to common human may be set as predetermined patterns, when such patterns are identified, some indexes indicating associated patterns are stored instead of storing the raw ECG or heart beating information. Please be noted that it is particularly helpful to prevent fatal accident occurred. If the mobile phone 14 is connected to the signal processor 1022, warning may be given to the user so as to alert the user to be cautious of potential risk, since some heart diseases may appear randomly and even without warning index.

Alternatively, the signal processor 1022 may refer to the clock time to determine how much physiological information is to be stored. For example, during the day time, more detailed information may be stored; during sleeping hours, less detailed information may be stored.

Generally, sampling rate is critical to power consumption and precision of collected information. When the signal processor is in low battery, the sampling rate may be decreased. Also, as mentioned above, the signal processor 1022 may contain various predetermined patterns. When certain pattern is identified, the sampling rate may be increased accordingly so as to capture more detailed information; for the other time, the sampling rate may be reduced so as to save power.

Besides, under proper circuit design, power consumption may be further reduced and under certain level, kinetic or energy-harvesting techniques such as power generated from body temperature may be applied for replacing traditional battery or as a supplementary power source. Other alternative ways like solar power may also be applied, too.

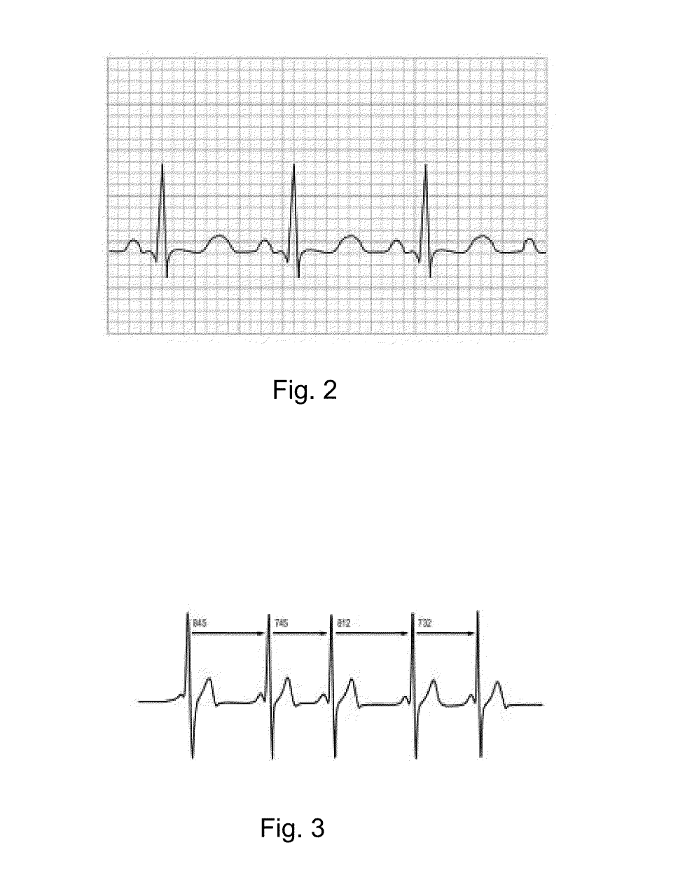

Furthermore, if the textile sensor 1021 is more sensitive, ECG (as illustrated in FIG. 2) and Heart Rate Variation (HRV, as illustrated in FIG. 3) may be both obtained. HRV refers to timing period variation among adjacent heart beats, and ECG refers to more complete wave form that contains more detailed information useful in various medical applications. As mentioned above, the signal processor 1022 may have different modes for ECG and HRV data collection. For example, when certain pattern of possible heart disease is identified, an ECG mode is activated to record more detailed waveform so it can be analyzed by a doctor to give medical advice. Otherwise, an HRV mode is activated and the signal processor 1022 only records HRV information and consumes relatively much lower energy.

The operating mode may be configured by the user via an external device like the mobile phone 14. Specifically, the mobile phone 14, when being connected to the signal processor 1022, may be used as an instant operating interface to control how the signal processor 1022 to operate. Furthermore, the patterns as mentioned above and/or other handling logic or program codes may be transmitted to the signal processor 1022 from an external device like the mobile phone 14. Alternatively, users may set the configuration of the signal processor 1022 via the mobile phone 14 and even when the mobile phone 14 is offline, no longer connected to the signal processor 1022, the signal processor 1022 may still be able to operate with the most updated configuration.

Collected physiological information after processed by the mobile phone 14 may be further stored in an external device 16 or uploaded to a cloud storage 18. Such physiological information, raw or processed format, may be provided to a computer 17 used by a doctor 171 or a fitness coach (not shown) via the external device 16 or the cloud storage 18. As mentioned above, the signal processor 1022 may be configured to identify certain patterns indicating higher possibility leading to certain heart disease, such information, e.g. ECG wave forms, is very helpful for finding fatal disease and preventing heart accident in advance. If the storage and power consumption is not a problem to the signal processor 1022, patterns may not need to be matched but raw ECG or HRV information may be provided to the doctor 171 for performing diagnosis.

The user wearing the shirt 101 may get instant feedback like hearing heart beat simulation sounds from the mobile phone 14, get music (song and/or album selection) that is adaptively adjusted by HRV information, and/or get instant warning from the mobile phone 14 when certain danger patterns are identified, smart coach advice by human or computer algorithms by analyzing HRV information, e.g. to run more vigorously or to take a rest now. There are a lot of other information types that may be provided to the user.

Alternatively, the mobile phone 14 may provide the user with audio information just like having a virtual fitness coach accompanied during exercise of the user. The content of the audio information may be even customized according to characteristics of the user. For example, it may be more beneficial to the users if they could do exercise and get the heart beat range within a target cardinal zone. The target cardinal zone may be different for people of different ages. Usually, it could be a percentage range, e.g. 60% to 80%, of a maximum heart rate, which might be the number ‘220’ minus the age of a user. For example, if a user is 40 years old, the maximum heart rate may be close to 180 heart beats per minute, and the target cardinal zone for this user may therefore fall in the range of 108 to 144 heart beats per minute.

For some medical research, it is recommended for a user to have exercise to stay in the target cardinal zone for two times each week and 30 minutes each time. When a user is wearing the shirt 101 and doing exercise, the mobile phone 14 with corresponding apps installed could inform the user with most updated status and even cheers up the user like, “you have running for 90 minutes, very well, and keep going for the rest 30 minutes.” Specific information may also be provided under certain user settings because every user may have different preferences. Some would like a dramatic encouragement, e.g. transforming bio-information and coach advice into an exciting and interesting theme music that changes during doing exercise. For example, a sound of cheers and clapping may be embedded into the audio content sent to the user who just achieves certain target. Furthermore, social community function may also be added. For example, two friends may run at different places, but are connected with their mobile phones via certain network. For example, John and Mary run at different places doing exercise, and the apps in their mobile phone 14 may check and establish communication so that the couple may run “together”, even sharing heart beat information to further encourage people to use the equipment and do more exercise, keeping them in better physical fitness status.

For some other users, they may want more scientific information, like actual heart beat numbers, or simulated heart beat sounds according to information collected from the shirt 101 that has sensors. And maybe even for some people who may have certain heart pain issues, a warning on time or detail recording on abnormal status of heart status may be given via designing associated app in the mobile phone or a corresponding electronic device.

Besides, when the collected physiological information or its derived information is uploaded to the cloud storage 18, another user may provide instant input to the user who wears the shirt 101. For example, two or more runners may run at the same time and share information via the cloud storage 18. Instant information may be provided among users to increase motivation of users to do exercise.

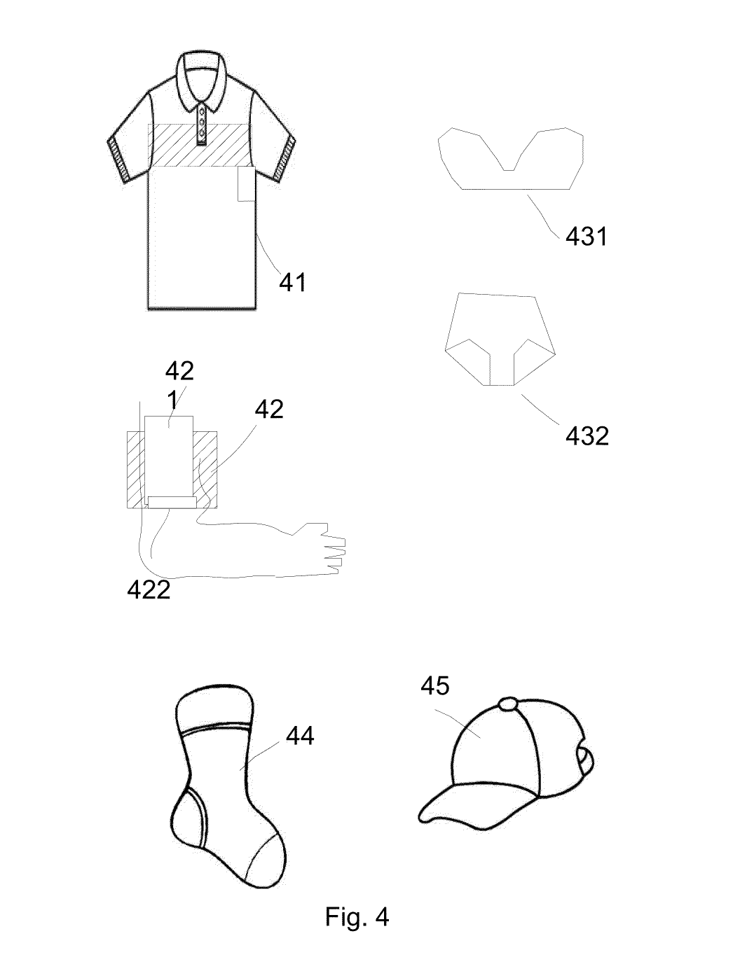

FIG. 4 illustrates various embodiments for disposing textile sensors like the textile sensor 1021 illustrated in FIG. 1. In addition to embed the textile sensor to a shirt 41, underwear 431, 432, an arm belt 42, a sock 44, a hat 45, a helmet (not shown), a wrist band (not shown) and a jewelry may be woven with textile sensors. In the case of an arm belt 42, which is popular for runners to carry their mobile phone during jogging or running, an interface device 422 connecting to the textile sensor woven in the arm belt 42 is provided to connect to a mobile phone 421 or an Walkman device like an iPod. In an embodiment, the interface device 422 has a mechanical socket for connecting to an external device like a mobile phone. The external device provides power to drive the interface device 422 and the textile sensor woven in the arm belt 42. In addition, most processing work may now be handled by the external device. Such design makes the arm belt 42 easily achieve water-proof, no need to change or charge battery, and low cost.

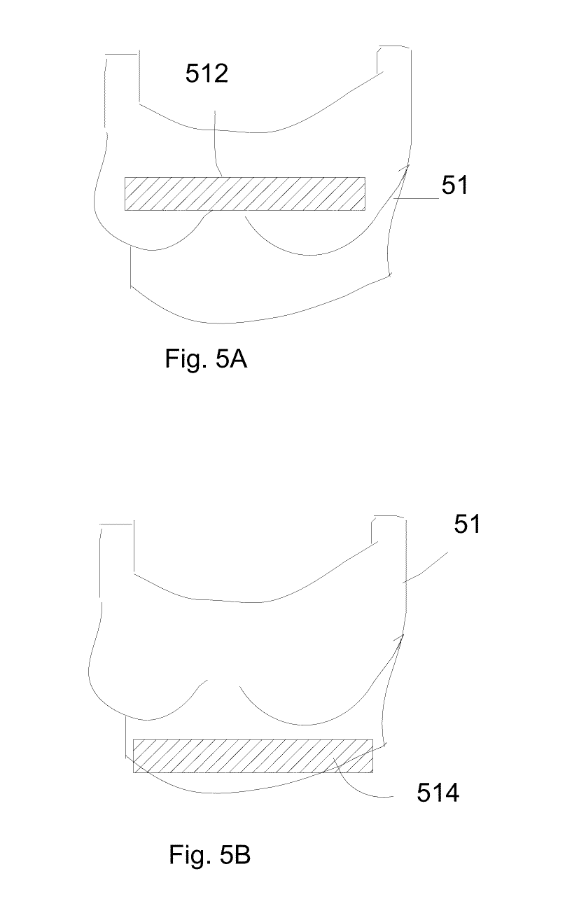

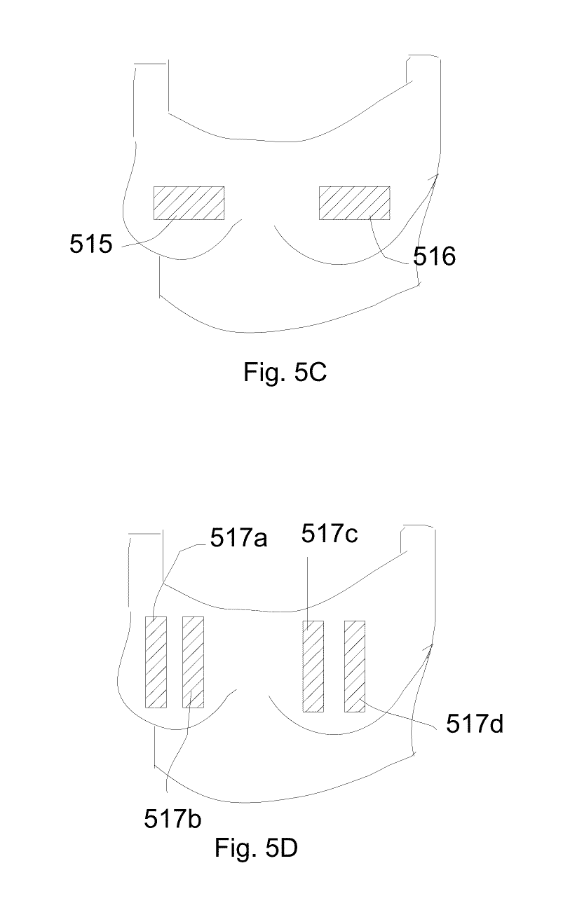

In some embodiments, bras are selected for embedding the textile sensors. Bras have cup structures that can be used for storing signal processor. FIG. 5A to FIG. 5D illustrate several examples for configuring the textile sensors and signal processors.

In FIG. 5A, the bra 51 may be a sports bra, a underwear bra, or a swimming bra. A textile sensor belt 512 is woven in the inner side of the bra 51 so as to contact the chest of a user to get heart beat information like ECG or HRV.

In FIG. 5B, the textile sensor belt 514 is placed below the cup to improve comfort of wearing while still being capable of collecting ECG and/or HRV information.

In FIG. 5C, two textile sensor segments 515, 516 are woven in the bra 51. In collecting ECG information, two or more electrodes are helpful for collecting better physiological signals. The two textile sensor segments 515, 516 are therefore used for collecting physiological signals upon the chest of a user for obtaining proper ECG information. In another aspect, two textile sensor segments 515, 516 may serve as redundant insurance. When one textile sensor segment is out of order, there is still another segment working for providing useful information. In an embodiment, corresponding modes may be configured to collect only signals from one segment to save power. Alternatively, signals from one segment may be used for correcting and/or adjusting information collecting form another segment like MIMO antennas help on collecting good quality Wi-Fi (IEEE 802.11) wireless signals.

In FIG. 5D, there are four textile sensor segments 517a, 517b, 517c, 517d embedded. Please be noted that there can be more arrangements of the textile sensor segments disposed on different places of a bra.

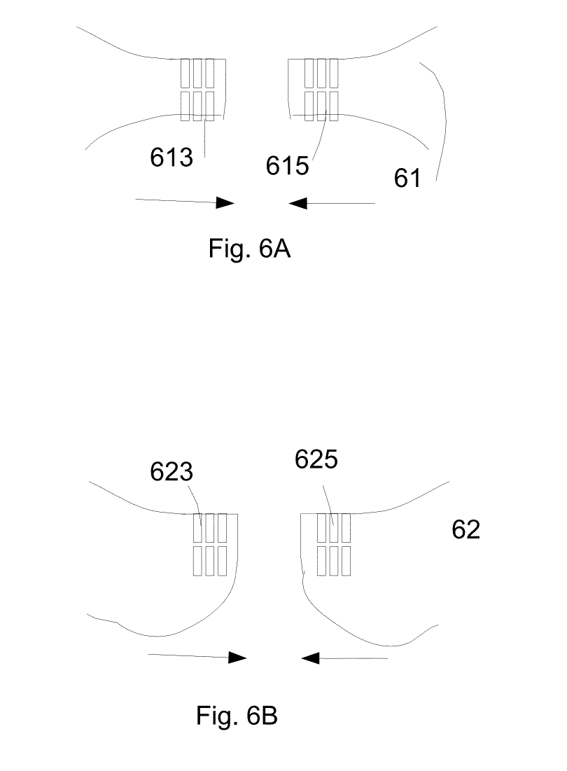

FIG. 6A illustrates backside of a bra. The buckle structures 613 and 615 of the bra 61 are used for fixing the bra 61 on user's body. In one embodiment, the coupling of the buckle structures 613 and 615 may serve as a switch to activate a signal processor (not shown). FIG. 6B shows another embodiment for placing the buckle structures 623 and 625 to the bra 62.

Sometimes, users may wear padding pieces inside their bras to look better. Therefore, in addition to directly weave textile sensors into bras as one whole body, textile sensors may be integrated with the padding pieces of a bra.

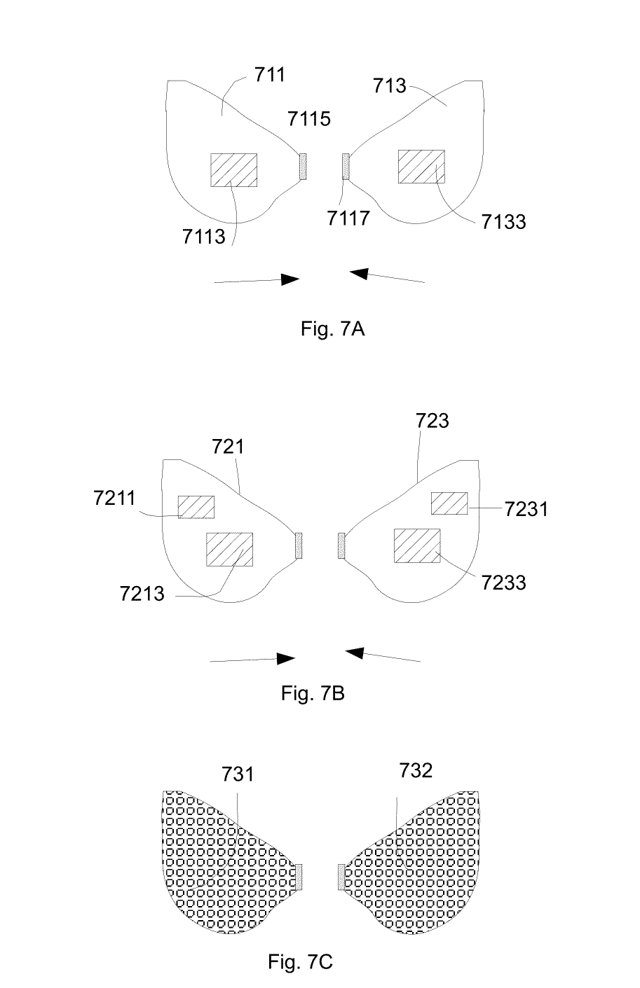

FIG. 7A illustrate one such padding piece to be worn inside a bra. Two padding bodies 711 and 713 are attached with textile sensor segments 7113 and 7133 as mentioned above. A pair of buckle structures 7115 and 7117 are used for connecting the two padding bodies 711 and 713. The padding bodies 711 and 713 may be made of silicon rubber, and the padding bodies 711 and 713 may contact human chest when being placed inside the bras. Alternatively, the padding bodies are made of common cotton or other textile fibers with padding materials filled inside.

FIG. 7B illustrates that in addition to embed the textile sensor segments 7213, 7233 in the padding bodies 721 and 723, signal processor 7211 and 7231 may also be embedded inside the padding bodies 721 and 723. Such configuration eliminates the need of an additional box attached to the bra and makes the product as a whole appear simpler and may be more appealing to some users.

FIG. 7C illustrates another embodiment to embed the textile sensors. As explained above, textile sensors may be conductive fibers woven into clothing. Therefore, when the padding bodies are silicon rubber for easily contacting human chest, conductive electrodes may be printed on surface of padding bodies while not covering the whole surface facing human chest so as to maintain contacting capability of the silicon rubber. In FIG. 7C, the small circles illustrate holes for exposing micro conductive electrodes contacting human chest for collecting ECG or HRV information. These micro conductive electrodes are interconnected inside the silicon rubber performing similar function as the textile sensor belt or segments as mentioned above.

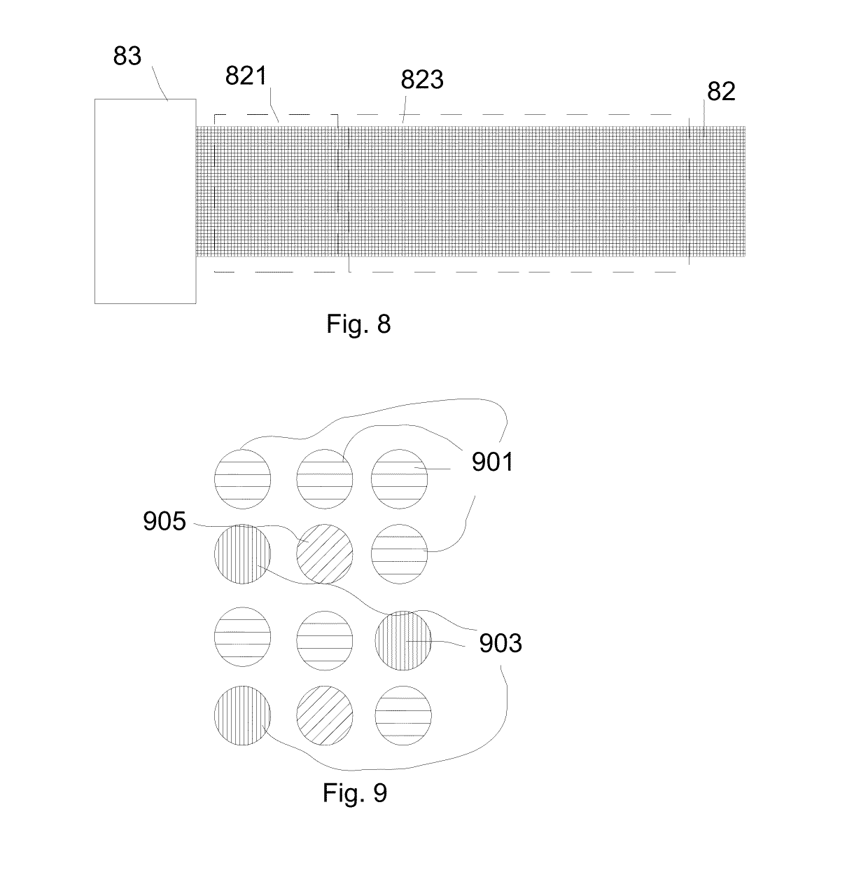

FIG. 8 illustrates a textile sensor belt 82 which can be woven into various clothing. There is a detection area 823 for disposing conductive fibers serving as electrodes for collecting information like ECG or HRV. There is also a transferring area 821 for transferring signals collected by the detection area 823 to a signal processor 83, which may be detachable or fixed to the textile sensor belt 82.

Because the textile sensor belt 82 is configured to contact human skin, it may be favorable to arrange some contact surface patterns to make users more comfortable while wearing such textile sensor belt 82.

FIG. 9 illustrates an embodiment for textile patterns on the contact surface of textile sensor belt 82. Conductive electrode fiber 903 do not occupy all surface and instead leave some areas for other functional fibers 901 and 905, e.g. moisture absorbing or warm keeping fibers. In addition to the patterns on the direct contact surface, other combination of functional layers like GoreTex® or other function layers may be integrated with above mentioned textile sensors to make wearers more comfortable for different purpose of clothing. For example, graphene or other materials, that have great performance on conducting currents, may be used for the conductive lines used in sensors and signal lines mentioned above. For example, silver is known for its capability to remove certain smell. When silver material is applied, it can achieve both conductivity and bad smell removing functions.

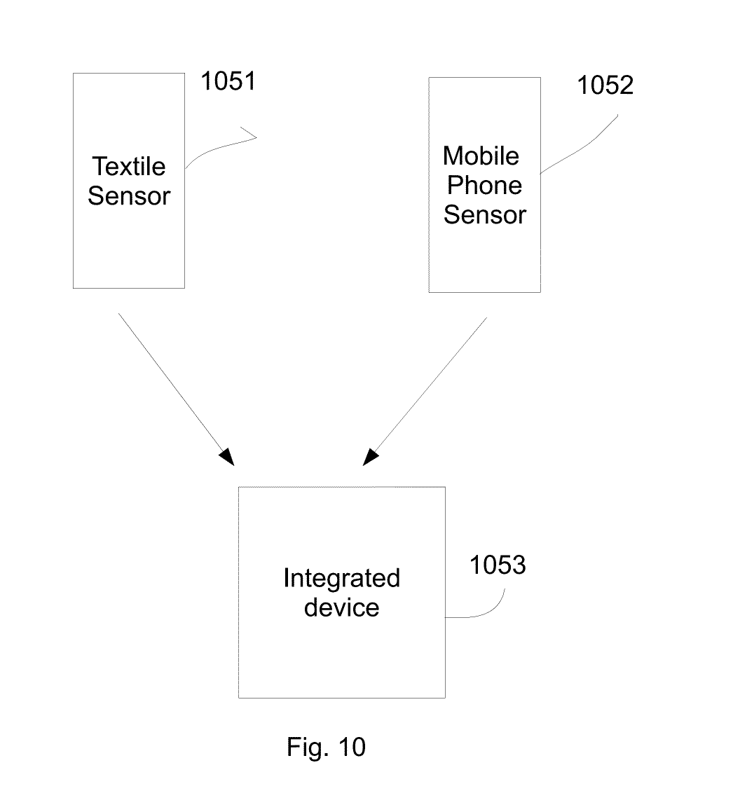

FIG. 10 illustrates textile sensor 1051 and mobile phone sensor 1052 may be further integrated by an integrated device 1053 to provide even more flexibility and uses of the sensor information [Jason to Nick: please modify FIG. 10 and change “sensor textile” to “textile sensor”]. When a user brings a mobile phone with motion sensors, GPS sensors, etc., such sensors generate useful information which may help describe the context situation. For example, current mobile phone may be used for identifying whether the user is moving and how the user is moving. Such motion information may be referenced together with the physiological information collected by textile sensor woven in the clothing. Information from external devices like a mobile phone may be provided to a signal processor in the clothing as mentioned above, in addition to just the textile sensor to provide information to the external devices. Such information could be very useful. For example, when a user stays still but his HRV changes dramatically, a warning could be provided to the user and the HRV information may be recorded for later diagnosis by a doctor. In other words, the information collected by a textile sensor, together with the information collected by input or by other means from an external device like a mobile phone, may be provided to the signal processor for further analysis to more intelligently respond and monitor users' physical condition.

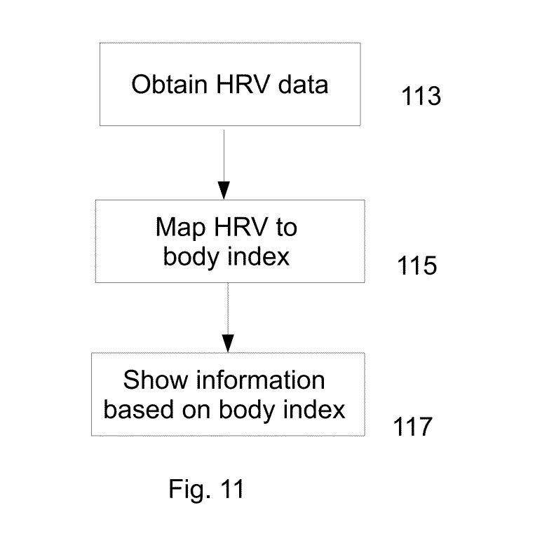

FIG. 11 illustrates a flow chart for using collected HRV or ECG information (step 113). Such collected information is mapped to body index like fitness index or health index or medical index or pressure index (Step 115). For example, HRV has certain relation to pressure felt by a user. When pressure situation is detected, a connected mobile phone may send a matching song to the user to make users feel more peaceful. Using such skills, HRV may even be used for selecting proper song or album to be played to achieve certain functions. For people who like to do exercise, HRV may also be used for indicating whether the users need to exercise harder now or stop for a rest. Such information is then shown or used (step 117).

In another embodiment, the shirts or other clothing with textile sensors mentioned above may be used as an identifier to identify a unique user, and thus may be used in various applications. For example, if the textile sensors are designed with higher sensibility, they can be used for collecting ECG. Since ECG patterns are different for different people, like fingerprints, a specific ECG pattern may be used to identify a unique user. In other words, the clothing with textile sensors may be used as an identifier of a user. Such capability may be integrated to unlock corresponding devices, like a mobile phone, or to inform a smart device in a living room, like a television, who the user is and associated response may be adopted to provide convenient service. In addition, advertisement or other applications when such information is obtained may also be achieved.

In another embodiment, software installed on a mobile phone as illustrated in the embodiment of FIG. 1 may be smartly designed to learn profiles and habits of users. For example, specific workouts or other products may be recommended to the users based on their behavior and historical data collected by the textile sensors.

In another embodiment, earrings, earphones with heartbeat sensors or other devices, in addition to the textile sensors, may be used to replace or to co-work with the textile sensors for enhancing the functions and applications of the textile sensor mentioned above.

Although the new protocol Bluetooth Low Energy (BTLE) is much more power efficient compared with previous Bluetooth protocols for handling device communication, there are several ways to apply on the communications between various heartbeat sensors and the external devices like mobile phones. For example, the mobile phone may record and analyze user habit and determines a power saving strategy from multiple operating modes accordingly. For example, a BTLE sensor device that detects heartbeat information may constantly send data to a mobile phone. This will drain the battery of the mobile phone quickly. The mobile phone with a designed App may record and learn patterns, routines, and context of a user to determine a corresponding operating mode for managing the BTLE sensor device and the mobile phone to listen and broadcast information. For example, when the mobile phone is in low battery, a less frequent communication between the BTLE sensor device and the mobile phone is determined. For another example, when the mobile phone learns a user is taking a two-hour workout by checking the user's schedule stored on the mobile phone, the signals generated by the motion sensors embedded in the mobile phone, or the user's past history records, the mobile phone may choose an operating mode in which the BTLE sensor does not constantly transmit heartbeat information to the mobile phone, except when the user pulls out the mobile phone to check real time information. When the BTLE sensor is not communicating with the mobile phone, heartbeat information is stored in the BLTE sensor and does not transmit to the mobile phone until the mobile phone requests such information.

This approach is not limited to BTLE protocol but may apply to other communication protocols, e.g. NFC, Wi-Fi, etc. In summary, an App may be designed to be installed on a mobile device like a mobile phone. The App collects information like motion sensor information, schedules, history records, current mobile use status, and/or battery status. The App dynamically determines an operating mode determining how the mobile phone communicates with a heartbeat sensing device like the textile sensor and the signal processor as mentioned above. An instruction corresponding to the determined operating mode may be transmitted to the heartbeat sensing device to change communication behavior of the heartbeat sensing device. For example, the communication behavior includes how frequently the heartbeat sensing device is to send collected data to the mobile phone.

In the above embodiments, the textile sensors are implemented as containing conductive fiber woven interleaved within insulation material fiber. It is noted that the textile sensors may also be implemented as electrical or optical sensors capable of detecting user's physiological signals.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention.

1. A bra device for a user to wear, comprising:
a textile sensor for detecting heart beat information;
a signal processor for processing the heart beat information and generating processed heart beat information; and
a transmitter for transmitting the processed heart beat information to an external device, the external device configured to transmit context information of the user to the signal processor as a reference for the signal processor to process the heart beat information.

2. The bra device of claim 1, wherein the context information comprises external sensor information gathered by a sensor of the external device.

3. The bra device of claim 2, wherein the external sensor information indicates movement of the user.

4. The bra device of claim 3, wherein the signal processor has a plurality of operating modes, and the processing sensor determines one from the operating modes to process the heart beat information based on the context information.

5. The bra device of claim 4, wherein the signal processor determines one from the operating modes according to whether the external device is wirelessly connected to the bra device.

6. The bra device of claim 4, wherein the signal processor determines one from the operating modes depending on current battery level of the bra device.

7. The bra device of claim 4, wherein the signal processor adopts different sampling rate to collect the heart beat information in different operating modes.

8. The bra device of claim 4, wherein the signal processor receives an instruction from the external device to determine one from the operating modes, the determined operating mode comprising frequency to communicate with the external device.

9. The bra device of claim 1, wherein the context information comprises schedule information indicating location where the user stays.

10. The bra device of claim 1, wherein the context information is gathered by the external device from a second external device.

11. The bra device of claim 1, wherein the external device is a mobile phone, and processing logic of the mobile phone is embedded from an app downloaded to the mobile phone from a network.

12. The bra device of claim 1, further comprising a storage for storing the heart beat information, wherein the signal processor detects an available capacity of the storage and stores only a pattern index instead of raw data of the heart beat information when the available capacity is below a threshold.

13. The bra device of claim 1, wherein when the signal processor finds the heart beat information matching a certain pattern, the signal processor switches to record the heart beat information in a different mode.

14. The bra device of claim 1, wherein when the certain pattern is related to a heart disease, generating a warning signal and sent it to the external device to alert the user.

15. The bra device of claim 1, wherein the heart beat information is categorized into a series of predetermined patterns, and only one or more indexes to the predetermined patterns are transmitted to the external device.

16. The bra device of claim 1, wherein the heart beat information comprises heart rate variation.

17. The bra device of claim 1, wherein the external device sends processing logic for processing the heart beat information on the signal processor to change processing manner for processing the heart beat information.

18. The bra device of claim 1, wherein the external device generates sounds corresponding to the received heart beat information.

19. The bra device of claim 1, further comprising a belt for carrying the textile sensor to contact the body of the user to collect the heart beat information.

20. The bra device of claim 1, wherein the textile sensor comprises a plurality of sensor parts, and a first signal collected from a first sensor part is used for adjust a second signal collected from a second sensor part.

21. The bra device of claim 1, wherein the textile sensor includes graphene material.
